(19) United States
(12) Patent Application Publication
(10) Pub. No.: US 2026/0185988 A1
(43) Pub. Date: Jul. 2, 2026

(54) METHODS FOR DETECTING TARGET COMPOUNDS AND USES THEREOF

(71) Applicant: Zhong Li, Short Hills, NJ (US)

(72) Inventor: Zhong Li, Short Hills, NJ (US)

(21) Appl. No.: 19/127,673

(22) PCT Filed: Nov. 8, 2023
(86) PCT No.: PCT/US2023/079128

§ 371 Date: May 6, 2025

Related U.S. Application Data
(60) Provisional application No. 63/423,634, filed on Nov. 8, 2022.

(51) Int. Cl. G01N 33/543; B01L 3/00; G01N 21/25; G01N 21/76; G01N 33/52; G01N 33/533; G01N 33/94

(52) U.S. Cl. CPC G01N 33/54326; B01L 3/5027; G01N 21/25; G01N 21/76; G01N 33/52; G01N 33/533; G01N 33/94; B01L 2200/0668; B01L 2400/043

(57) ABSTRACT

The present invention relates to methods for detecting target compounds in a sample quickly. A method for detecting one or more target compounds in a sample is provided. The method comprises mixing a sample with a solution that contains magnetic labels covered with one or more capturing probes; forming complexes of the one or more target compounds and the one or more corresponding capturing probes; washing the magnetic labels in a solution; applying a magnetic field to bring the magnetic labels in proximity with one or more detecting probes to form one or more super-complexes of detecting probe and corresponding existing complexes of capturing probe and target compound; isolating the super-complexes; and detecting the presence of the super-complexes.

### CROSS-REFERENCE TO RELATED APPLICATION

This application claims priority to U.S. Provisional Application No. 63/423,634, filed on Nov. 8, 2022, which is herein incorporated by reference in its entirety.

### FIELD OF THE INVENTION

The present invention relates generally to methods for fast, qualitative/semi-quantitative/quantitative, and multiplex detection of target compounds.

### BACKGROUND OF THE INVENTION

Overdose from both licit and illicit drugs has been an increasingly common cause of death in persons fifteen to seventy years of age. After a suspected drug death, a major objective at autopsy is to determine whether any drugs measured in the decedent have played a role in the decedent's cause of death.

The 2020 National Survey on Drug Use and Health (NSDUH) estimated that more than 40 million Americans aged 12 or older had a substance use disorder (SUD) in the past year. Although treatable, SUDs can lead to significant problems in all aspects of a person's life, including death from drug overdose. Data collected by the Centers for Disease Control and Prevention (CDC) and the National Institute on Drug Abuse (NIDA) showed that over one million individuals in the US have died from drug overdose from 1999 to 2021 at an accelerating rate over recent years. The increasing death toll and the increase in the variety of abused substances in our society have strained our medical resources and hinder the efforts to identify threat in a timely manner and to develop faster and targeted response to treat drug overdose and prevent death.

Suspected drug overdoses represent more than one in six death investigations currently. Those death investigations require comprehensive toxicology analysis to identify the drugs involved and determine the cause of death, which currently takes days or week to obtain result at growing cost. The backlog at many coroner and medical examiner offices may be months long and get worse by surging number of cases, especially death involving synthetic opioids such as Fentanyl. Major reasons for the backlog include the cost of instrument, technical staffing, and laboratory costs for a comprehensive toxicology analysis.

Solutions have been proposed in the prior art patent literature for post-mortem toxicology screening. For example, U.S. Pat. No. 10,267,811 describes a rapid, sensitive method for forensic drug testing in a post-mortem subject using oral fluid collected from the post-mortem subject. The method comprises collecting a sample of oral fluid from a post-mortem subject, analyzing the oral fluid sample qualitatively to detect the presence of one or more non-naturally occurring drugs, analyzing the oral fluid sample quantitatively to determine concentration of the one or more non-naturally occurring drugs in the post-mortem subject, and identifying the one or more non-naturally occurring drugs in the post-mortem subject. The detection and quantification in oral fluid is more sensitive and faster than detection and quantification of the non-naturally occurring drugs in blood, urine, bile, and liver tissue collected from the same post-mortem subject. Further, the qualitative and quantitative results are obtained in as little as three hours.

The above disclosure describes gas chromatography-mass spectroscopy (GC-MS) and liquid chromatography-mass spectrometry (LC-MS). The equipment cost for LC-MS is between $350,000 and $500,000. These sophisticated instruments need well-trained technical experts to operate. Understandably, the high cost to set up and run a GC-MS or LC-MS toxicology lab forces most coroner and medical examiner offices to submit postmortem biological samples to third-party dedicated toxicology testing labs, resulting in significant backlog in those labs.

Immunoassays using are also known in the patent literature. Immunoassays have been used extensively in toxicology screening, mostly using urine samples. It has the benefits of low cost and ease of use, and can produce results in minutes. Automated immunoassays are available with dedicated instruments. However, immunoassays in general are limited in the range and specificity of tested drugs and are considered to be qualitative, not quantitative.

For example, U.S. Pat. No. 10,145,843 describes an apparatus and method for rapid determination of analytes in liquid samples by immunoassays incorporating magnetic capture of beads on a sensor, capable of being used in the point-of-care diagnostic field, including, for example, use at accident sites, emergency rooms, in surgery, in intensive care units, and also in non-medical environments. This method comprises (a) mixing magnetically susceptible beads coated with a capturing antibody to an analyte with a sample containing the analyte and a signal antibody to form a sandwich on said beads; (b) applying the mixture to the immunosensor; (c) magnetically localizing and retaining at least a portion of said beads on the electrode; (d) washing the unbound sample from the electrode; (e) exposing the signal antibody of the sandwich to a signal generating reagent; and (f) measuring a signal from the reagent at the electrode.

Nevertheless, prior art systems, devices, and methods suffer from limitations including high cost, long lag time, and inconvenience. Solutions are urgently needed not only for postmortem toxicology screening, but also for routine drug screening. Furthermore, a fast, low-cost, and quantitative solution may also be used in fresh/waste water monitoring, infectious disease monitoring, molecular diagnostics, etc.

### SUMMARY OF THE INVENTION

The present invention relates to methods for detecting target compounds in a sample quickly.

A method for detecting one or more target compounds in a sample using a pair of probes for each target compound and one type of magnetic label is provided. The method comprises:
mixing a sample with a first solution that contains magnetic labels covered with one or more capturing probes, the one or more capturing probes being specific to one or more target compounds to be detected in the sample, the one or more capturing probes being immobilized exclusively on individual magnetic labels of the magnetic labels;
forming, in the first solution, complexes of the one or more target compounds and the one or more corresponding capturing probes;
washing the magnetic labels in a second solution;
mixing the magnetic labels in a third solution;
applying a magnetic field to bring the magnetic labels in proximity with one or more detecting probes, the one or more detecting probes being specific to one or more target compounds to be detected in the sample, to form one or more super-complexes of detecting probe and corresponding existing complexes of capturing probe and target compound;
removing the magnetic field;
isolating the super-complexes; and
detecting the presence of the super-complexes.

A method for detecting one or more target compounds in a sample using a single probe for each target compound and two types of magnetic label is provided. The method comprises:
mixing in a first solution large magnetic labels covered with one or more capturing probes, the one or more capturing probes being specific to one or more target compounds to be detected in the sample, the one or more capturing probes being immobilized exclusively on individual large magnetic labels of the large magnetic labels, with small magnetic labels covered with one or more target compounds to be detected in the sample, the one or more target compounds being immobilized exclusively on individual small magnetic labels of the small magnetic labels;
forming, in the first solution, large magnetic labels whose surface is covered with small magnetic labels;
mixing a sample in a second solution containing large magnetic labels covered with small magnetic labels, wherein target compounds in the sample compete with target compounds on the small magnetic labels to bind to capturing probes on the large magnetic labels to release into solution the corresponding small magnetic labels from the large magnetic labels;
washing the large and small magnetic labels in a third solution;
mixing the large and small magnetic labels in a fourth solution;
filtering the large magnetic labels from the released small magnetic labels;
applying a magnetic field to bring the small magnetic labels in proximity with one or more detecting probes to form one or more complexes of detecting probe and corresponding target compound;
removing the magnetic field;
isolating the super-complexes; and
detecting the presence of the super-complexes.

The methods further comprise providing a surface with the one or more detecting probes attached thereto.

The sample may include whole blood. The sample may be postmortem whole blood.

The one or more detecting probes may be arranged in a linear array. The one or more detecting probes may be arranged in a planar array. The one or more detecting probes include antibodies. The one or more capturing probes include antibodies. The one or more capturing probes include aptamers.

The one or more target compounds are under the drug classes of barbiturates, benzodiazepines, cannabinoids, dissociatives, cocaine, opioids, and sympathomimetics amines.
detecting includes at least one of detecting at least one of fluorescence, luminescence, giant magnetoresistance (GMR), and color.

The one or more complexes include at least one of the binding combinations biotin-streptavidin, antigen-antibody, and nucleotide double helix.

A system for detecting one or more target compounds in a sample is provided, comprising:
a microfluidic subsystem configured to mix a sample with a first solution that contains magnetic labels covered with one or more capturing probes, the one or more capturing probes being specific to one or more target compounds to be detected in the sample, the one or more capturing probes being immobilized exclusively on individual magnetic labels of the magnetic labels, form, in the first solution, complexes of the one or more target compounds and the one or more corresponding capturing probes, wash the magnetic labels in a second solution, and mix the magnetic labels in a third solution;
a magnetic field generator configured to bring the magnetic labels in proximity with one or more detecting probes, the one or more detecting probes being specific to one or more target compounds to be detected in the sample, to form one or more super-complexes of detecting probe and corresponding existing complexes of capturing probe and target compound;
the microfluidic subsystem configured to isolate the super-complexes; and
a detection subsystem configured to determine the presence and quantity of the super-complexes.

A system for detecting one or more target compounds in a sample following an alternative workflow is provided, comprising:
a microfluidic subsystem configured to mix a sample with a first solution that contains large magnetic labels covered with small magnetic labels, wherein target compounds in the sample compete with target compounds on the small magnetic labels to bind to capturing probes on the large magnetic labels to release into solution the corresponding small magnetic labels from the large magnetic labels, wash the large and small magnetic labels in a second solution, and mix the magnetic labels in a third solution;
a magnetic field generator and a filter configured to bring only the released small magnetic labels in proximity with one or more detecting probes, the one or more detecting probes being specific to one or more target compounds to be detected in the sample, to form one or more complexes of detecting probe and corresponding target compound;
the microfluidic subsystem configured to isolate the super-complexes; and
a detection subsystem configured to determine the presence and quantity of the super-complexes.

### BRIEF DESCRIPTION OF THE DRAWINGS

FIG. 1 shows a flow chart of a fast and multiplex method to detect one or more target compounds in a sample. The flow chart describes the steps in a non-competitive workflow.

FIG. 2 shows two states of a test cartridge in both non-competitive and competitive workflows. The left image illustrates that the test chamber of a test cartridge is partially filled with reagent 203. The inner top flat surface 202 of the test chamber, which is attached with a spatially arranged 2D array of probes, has no contact with reagent 203. The right image illustrates that the test chamber of a test cartridge 208 is fully filled with reagent 205. The inner top flat surface 206 of the test chamber has full contact with reagent 205. Magnet Y is represented as 201 and 204 in the left and right images, respectively. Magnet X is represented as 207 and 209 in the left and right images, respectively.

FIG. 3 shows a flow chart of a fast and multiplex method to detect one or more target compounds in a sample. The flow chart describes the steps in a competitive workflow.

FIG. 4 shows components of a device that may carry out either the method with the non-competitive workflow or the method with the competitive workflow to detect target compounds in a sample.

### DETAILED DESCRIPTION OF THE INVENTION

The present invention relates to methods for detecting target compounds in a sample quickly. In one embodiment, it is a fast, semi-quantitative, array-based multiplex immunoassay for postmortem toxicology screening. In another embodiment, it is a fast, quantitative, array-based multiplex immunoassay for postmortem toxicology screening.

The present invention provides a first method, a non-competitive workflow, for detecting one or more target compounds in a sample using a pair of probes for each target compound and one type of magnetic label. One of the probe pair for each target compound, the capturing probe, may be attached to a magnetic label. The other probe for each target compound, the detecting probe, may be attached to a solid surface at one or more spatially defined locations. The capturing probe may bind to the corresponding target compound to form a complex. The capturing probe and the detecting probe pair may both bind to the corresponding target compound to form a complex. The method comprises mixing a sample with a first solution that contains magnetic labels covered with one or more capturing probes, the one or more capturing probes being specific to one or more target compounds to be detected in the sample, the one or more capturing probes being immobilized exclusively on individual magnetic labels of the magnetic labels; forming, in the first solution, complexes of the one or more target compounds and the one or more corresponding capturing probes; washing the magnetic labels in a second solution; mixing the magnetic labels in a third solution; applying a magnetic field to bring the magnetic labels in proximity with one or more detecting probes, the one or more detecting probes being specific to one or more target compounds to be detected in the sample, to form one or more super-complexes of detecting probe and corresponding existing complexes of capturing probe and target compound; removing the magnetic field; isolating the super-complexes; and detecting the presence of the super-complexes.

The present invention provides a second method, a competitive workflow, for detecting target compounds in a sample using a single probe for each target compound and two types of magnetic label. The probe for each target compound may be attached to a magnetic label. The probe for each target compound may also be attached to a solid surface at one or more spatially defined locations. Each target compound may be attached to a magnetic label. The method comprises mixing in a first solution large magnetic labels covered with one or more capturing probes, the one or more capturing probes being specific to one or more target compounds to be detected in the sample, the one or more capturing probes being immobilized exclusively on individual large magnetic labels of the large magnetic labels, with small magnetic labels covered with one or more target compounds to be detected in the sample, the one or more target compounds being immobilized exclusively on individual small magnetic labels of the small magnetic labels; forming, in the first solution, large magnetic labels whose surface is covered with small magnetic labels; mixing a sample in a second solution containing large magnetic labels covered with small magnetic labels, wherein target compounds in the sample compete with target compounds on the small magnetic labels to bind to capturing probes on the large magnetic labels to release into solution the corresponding small magnetic labels from the large magnetic labels; washing the large and small magnetic labels in a third solution; mixing the large and small magnetic labels in a fourth solution; filtering the large magnetic labels from the released small magnetic labels; applying a magnetic field to bring the small magnetic labels in proximity with one or more detecting probes to form one or more complexes of detecting probe and corresponding target compound; removing the magnetic field; isolating the super-complexes; and detecting the presence of the super-complexes.

The term “target compound” as used herein refers to a naturally occurring or man-made artificial material, for example, recombinantly or chemically, comprising a target biological molecule, chemical compound, cell, or tissue. Examples of target compound include small molecules, controlled substances, drugs, polynucleotides, polypeptides, polysaccharides, antibodies, monoclonal antibodies, cell membrane receptors, cofactors, sugars, lectins, cells, and cellular membrane. A target compound may be isolated from a sample. A target compound may be detected in a sample.

Target compounds may be compounds listed in Tables 1 and 2 in the 2021 ANSI/ASB Standard for the Analytical Scope and Sensitivity of Forensic Toxicological Testing of Blood in Medicolegal Death Investigations. They may be under the drug classes of barbiturates, benzodiazepines, cannabinoids, dissociatives, cocaine, opioids, and sympathomimetics amines. They may include, for example, Acetone, Isopropanol, Ethanol, Methanol, 10-OH-carbazepine, Carbamazepine, Gabapentin, Lamotrigine, Levetiracetam, Pregabalin, Phenytoin, Primidone, Topiramate, Amitriptyline, Bupropion, Citalopram, Clomipramine, Desipramine, Doxepin, Duloxetine, Fluoxetine, Imipramine, Mirtazapine, Nortriptyline, O-desmethylvenlafaxine, Paroxetine, Sertraline, Trazodone, Venlafaxine, Chlorpheniramine, Diphenhydramine, Doxylamine, Hydroxyzine, Methorphan, Promethazine, 9-hydroxyrisperidone, Risperidone, Chlorpromazine, Clozapine, Olanzapine, Quetiapine, Butalbital, Pentobarbital, Phenobarbital, Secobarbital, 7-aminoclonazepam, Alprazolam, Clonazepam, Lorazepam, Zolpidem, Diazepam, Nordiazepam, Oxazepam, Temazepam, THC, THC-COOH, COHb, Ketamine, Phencyclidine, Cocaine, Cocaethylene, Benzoylecgonine, Cyclobenzaprine, Carisoprodol, Meprobamate, Buprenorphine, Fentanyl, 6-acetylmorphine, Oxymorphone, Codeine, Hydrocodone, Hydromorphone, Morphine, Oxycodone, Methadone, Tramadol, Acetaminophen, Salicylates, Amphetamine, Methamphetamine, Methylenedioxyamphetamine (MDA), and Methylenedioxymethamphetamine (MDMA).

The term “sample” as used herein refers to a naturally occurring material, a man-made material, or a mixture of naturally occurring material and man-made material. A sample may be any kind of liquid or liquefiable material that is suspected to comprise one or more target compounds. Examples of the samples are biological sample and environmental sample. A biological sample is obtained from a biological source, for example, an organism such as a microorganism, animal or plant, preferably a mammal, more preferably a human. Examples of biological samples include blood, serum, ascites fluid, cerebrospinal fluid, amniotic fluid, synovial fluid, pleural fluid, saliva, sputum, stool, urine, semen, tissue, biopsies, swabs, and the like from human and non-human sources. An environmental sample is obtained from an environmental source such as air, water, soil, and environments exposed to extremes of conditions (e.g., temperature or pressure). Environmental samples may include samples from industrial processes. A sample may be mixed with a solution to produce another sample. One embodiment utilizes a whole blood sample in which target compounds may be detected.

The term “probe” as used herein refers to an agent capable of binding to a target compound specifically. The term “capturing probe” as used herein refers to an agent capable of binding to a target compound specifically in a sample and facilitating target compound enrichment. An example of capturing probe is capturing antibody, The term “detecting probe” as used herein refers to an agent capable of binding to a target compound specifically and facilitating target compound detection and/or quantification. An example of detecting probe is detecting antibody.

The terms “polypeptide” and “protein” are used herein interchangeably, and refer to a polymer of amino acids of any length. The polypeptide may have about 5-5000, 5-1000, 10-100, 10-50, 10-40, or 10-20 amino acids. An amino acid may be modified from a naturally occurring amino acid by, for example, glycosylation or phosphorylation. In some embodiments, one or more amino acids in the target polypeptide may be modified to stabilize or destabilize the binding complex formation between the target polypeptide and the polypeptide probe, or to enhance the specificity of binding with the polypeptide probe.

Where the target compound comprises a target small molecule or a polypeptide, the probe may be a polypeptide probe that binds specifically to the target small molecule or polypeptide. The polypeptide probe may comprise about 5-5000, 5-1000, 10-100, 10-50, 10-40, or 10-20 amino acids, The polypeptide probe may be an antibody or a single-chain variable fragment that binds specifically to the target polypeptide.

The term “antibody” as used herein includes whole antibodies, antigen binding fragments (or antigen-binding portions), and single chains thereof. A whole antibody refers to a glycoprotein typically having two heavy chains and two light chains, and includes an antigen binding portion. The term “antigen binding portion” of an antibody as used herein refers to one or more fragments of the antibody that retain the ability of specifically binding to an antigen. The term “single-chain variable fragment” of an antibody as used herein refers to a fusion protein of the variable regions of the heavy and light chains of the antibody, connected with a short linker peptide, for example, of about 20-25 amino acids, that retains the ability of specifically binding to an antigen.

The term “polynucleotide” as used herein refers a polymer of nucleotides of any length. A polynucleotide may be an oligonucleotide having fewer than 1000 nucleotides. The polynucleotide may be any type of single stranded of DNA, cDNA, RNA, or a combination or derivative thereof. The polynucleotide may be a peptide nucleic acid (PNA). The polynucleotide may be linear or circular, preferably linear. A polynucleotide may have about 5-2000, 5-100, 5-50, 8-50, 10-40, 20-40 or 8-25 nucleotides, for example, about 8, 24 or 50 nucleotides.

Where the target compound comprises a target small molecule or a polynucleotide, the probe may be a polynucleotide probe having a nucleotide sequence that binds specifically to the target small molecule or is complementary with the nucleotide sequence of the target polynucleotide, respectively. The polynucleotide probe may be an oligonucleotide having about 5-2000, 5-100, 5-50 or 10-25 nucleotides, and may be linear or circular, preferably linear. A polynucleotide probe may be an aptamer.

The term “magnetic label” as used herein refers to a moiety attached to a probe, directly or indirectly, that is capable of moving the probe in response to a magnetic field, The magnetic label may be a particle (e.g., bead) comprising a magnetic material, and may be magnetic, paramagnetic, superparamagnetic, ferromagnetic, or diamagnetic. The particle may be made of any inert material known in the art, for example, plastic, metal, glass and ceramic, and have of any shape, preferably round. The magnetic particle may have a diameter ranging from about 1 nm to about 100 μm, from about 500 nm to about 10 μm, from about 750 nm to about 5 μm, or from about 900 nm to about 2 μm, preferably about 1 μm.

The terms “large magnetic label” and “small magnetic label” as used herein refer to two types of magnetic labels with different sizes. The large magnetic label is larger in size than the small magnetic label. An example of large magnetic label is large superparamagnetic bead. An example of small magnetic label is small superparamagnetic bead. A large superparamagnetic bead is larger in size than a small superparamagnetic bead.

The probe may be attached to a magnetic label via a linker. The linker may be any material capable of binding the probe specifically, but not the target compound, for example, a capture probe, biotin-streptavidin, Acrydite-SH, COOH—NH2, NH2—COOH, OH—BrCN, disulfide bond, Hydrazide-ligand with oxidized carbohydrate, protein A-antibody such as IgG, anti-mouse IgG-mouse IgG, or cleavable conjugating molecule. A capture probe may be a polynucleotide having a nucleotide sequence complementary with at least a portion of the nucleotide sequence of the polynucleotide probe, and capable of forming a hybrid with the polynucleotide probe on the surface of the magnetic particle. The portion of the polynucleotide probe that is not hybridized to the capture probe may hybridize with the target compound. In one embodiment, the probe may be biotinylated, and the magnetic particle may be a streptavidin-coated superparamagnetic bead (e.g., Dynabeads MyOne beads of Thermo Fisher Scientific, or ProMag beads from Bangs Lab).

The surface may be a solid surface. The solid surface may be any solid surface suitable for attachment of the biomarker. The solid surface may be made from one or more materials selected from the group consisting of polymers, plastics, resins, polysaccharides, silica or silica-based materials, carbon, metals, inorganic glasses, and membranes. The solid surface may be flat or curved. The solid surface may be separated into different regions, for example, wells. The solid surface may also be in the form of beads, resins, gels, microspheres, or other geometric configurations,

A probe may be attached to the solid surface directly via a covalent or non-covalent bond, preferably a covalent bond. The probe may also be attached to the solid surface indirectly, for example, via a linker, which may be cleavable. For example, the linker may be selected from the group consisting of biotin-streptavidin, Acrydite-SH, COOH—NH2, NH2—COOH, OH—BrCN, disulfide bond, Hydrazide-ligand with oxidized carbohydrate, protein A-antibody such as IgG, anti-mouse IgG-mouse IgG, and cleavable conjugating molecules.

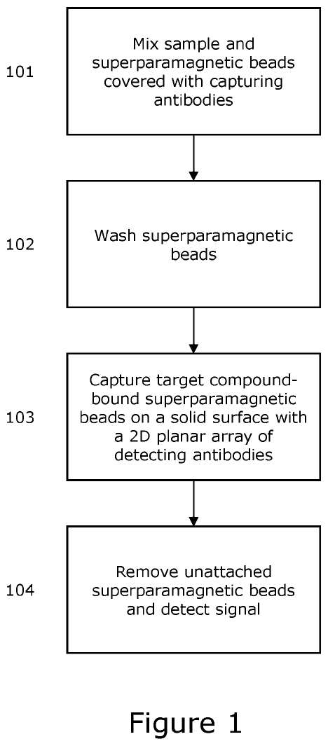

In one embodiment, as shown in FIG. 1, the non-competitive workflow is described as a fast, semi-quantitative, and multiplex immunoassay for the purpose of detecting controlled substances in postmortem whole blood.

In Step 101, aimed to be completed within 20 minutes, a small volume (for example, 100 ul) of postmortem whole blood is mixed with Reagent A (for example, 300 ul) that contains superparamagnetic beads covered with one or more capturing antibodies. Each superparamagnetic bead is covered with a capturing antibody that is specific to a target compound, such as Fentanyl, to be analyzed in the postmortem toxicology screening.

In Step 102, aimed to be completed within five minutes, the superparamagnetic beads are washed one or more times in Reagent B (for example, 300 ul) and then resuspended in Reagent C (for example, 500 ul).

In Step 103, aimed to be completed within 20 minutes, the superparamagnetic beads in Reagent C are brought under a magnetic field to be in close contact with solid surface-bound detecting antibodies that are spatially arranged in a 2D planar array.

In Step 104, aimed to be completed within one minute, upon the removal of the magnetic field, Reagent C is removed along with unattached superparamagnetic beads. An image of the solid surface is captured with a camera of a smartphone and then processed by a smartphone app to produce result.

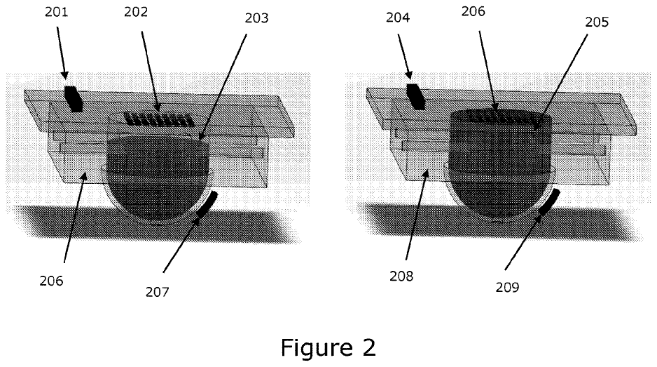

Reagent A may be a buffered solution containing superparamagnetic beads covered with one or more capturing antibodies. Each bead may be attached with one capturing antibody that is specific to a target compound listed in Tables 1 and 2 in the 2021 ANSI/ASB Standard for the Analytical Scope and Sensitivity of Forensic Toxicological Testing of Blood in Medicolegal Death Investigations. After postmortem whole blood is mixed with Reagent A in a partially filled test chamber of a test cartridge (FIG. 2, left image), target compounds in the whole blood may be captured by corresponding capturing antibodies and attached to corresponding superparamagnetic beads. The test chamber may be only partially filled so that Reagent A won't reach the inner top flat surface of the test chamber. Movement of Magnet X (FIGS. 2, 207 and 209) may facilitate target compound capture by creating superparamagnetic bead movement within Reagent A while the test cartridge remains motionless.

Once Step 101 is completed, Magnet X immobilizes superparamagnetic beads on a surface of the test chamber while the liquid may be completely drained from the chamber into the waste container. Subsequently in Step 102, Reagent B, which is a wash solution to remove unbound molecules from the test chamber and from beads, including excess of target compounds, may be injected into the chamber to wash over the partially filled test chamber and beads. The test chamber may be again only partially filled so that Reagent B won't reach the inner top flat surface of the test chamber. The resuspension of beads in Reagent B may be facilitated by Magnet X. The beads may be washed by Reagent B one or more times before they are resuspended in Reagent C.

Similar to Reagent A, Reagent C may be a buffered solution to facilitate antibody-antigen binding. In Step 103, after the test chamber may be fully filled with Reagent C (FIG. 2, right image) and superparamagnetic beads are fully resuspended, Magnet Y (FIGS. 2, 204 and 201), which is on top of the test cartridge but positioned at a distance to the test chamber, may move across the top surface to draw superparamagnetic beads to the inner top flat surface of the test chamber that has not been exposed to Reagent A or Reagent B but is now exposed to Reagent C due to the design of the test chamber and the volume of Reagents used (FIG. 2, right image). Lateral movement of Magnet Y may facilitate local enrichment of superparamagnetic beads near detecting antibodies attached to designated locations in a 2D array on the inner top flat surface of the test chamber. Target compounds that are captured by capturing antibodies that are attached to superparamagnetic beads may bind to corresponding detecting antibodies attached to the inner top flat surface of the test chamber. As a result, corresponding superparamagnetic beads may be attached at the locations for corresponding detecting antibodies.

In Step 104, Magnet Y may be moved back to its original position and Reagent C may be withdrawn completely from the test chamber along with unbound superparamagnetic beads. The number of superparamagnetic beads retained at designated locations of the solid surface may correlate in a qualitative, semi-quantitative, or quantitative manner with the level of corresponding target compound in the sample. The presence of bound superparamagnetic beads darkens the corresponding positions in the 2D array and the resulting 2D pattern of light/dark mosaic may be subsequently captured by a camera of a smartphone and analyzed by an image processing algorithm implemented in a smartphone app. The optic-based spatial detection of signals allows (1) the use of a cheaper instrument in comparison to fluorescence or luminescence-based instruments and (2) scalable parallel signal detection.

A test cartridge may have multiple test chambers so that multiple samples can be tested concurrently, or a single sample can be tested sequentially with different sets of antibodies to minimize cross-reactivity and potential conflict of binding conditions among antibodies.

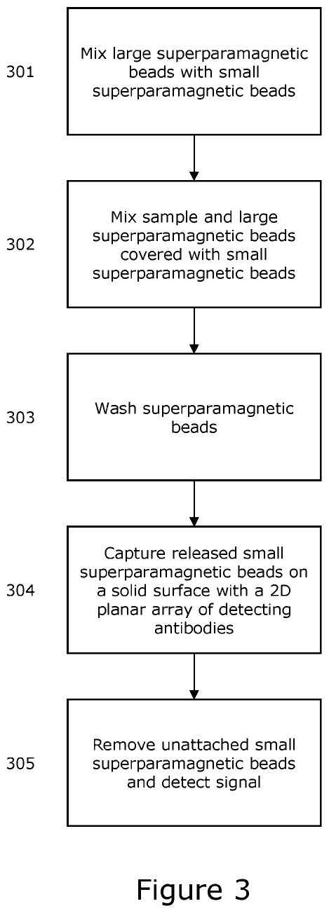

In another embodiment, as shown in FIG. 3, the competitive workflow is described as a fast, semi-quantitative, and multiplex immunoassay for the purpose of detecting controlled substances in postmortem whole blood.

Two types of superparamagnetic beads, large superparamagnetic beads and small superparamagnetic beads, may be used in the competitive workflow. One or more capturing antibodies may be attached to large superparamagnetic beads. Each large superparamagnetic bead may be covered with a capturing antibody that is specific to a target compound to be analyzed in the postmortem toxicology screening. One or more target compounds or conjugates of target compounds may be attached to small superparamagnetic beads. Each small superparamagnetic bead may be covered with a target compound or a conjugate of a target compound to be analyzed in the postmortem toxicology screening.

In Step 301, large superparamagnetic beads with attached capturing antibodies may be mixed with small superparamagnetic beads with attached target compounds in Reagent D so that small superparamagnetic beads may cover the surface of corresponding large superparamagnetic beads through target compound-capturing antibody binding. Unbound small superparamagnetic beads may be washed away with Reagent B. Large superparamagnetic beads that are covered with small superparamagnetic beads may be resuspended in a buffered solution to make Reagent E.

In Step 302, aimed to be completed within 20 minutes, a small volume (for example, 100 ul) of postmortem whole blood may be mixed with Reagent E (for example, 300 ul) containing large superparamagnetic beads that are covered with small superparamagnetic beads. Target compounds, if exist in the postmortem whole blood, compete with target compounds attached to the small superparamagnetic beads to bind to capturing antibodies on the large superparamagnetic beads. As a result, depending on the relative abundance of target compounds in postmortem whole blood, portions of small superparamagnetic beads that are bound to large superparamagnetic beads may be detached from the surface of large superparamagnetic beads.

In Step 303, aimed to be completed within five minutes, large and small superparamagnetic beads are washed one or more times in Reagent B (for example, 300 ul) and then resuspended in Reagent F (for example, 500 ul).

In Step 304, aimed to be completed within 20 minutes, the small superparamagnetic beads that are detached from the large superparamagnetic beads, not the large superparamagnetic beads, may be brought under a magnetic field to be in close contact with solid surface-bound detecting antibodies that are spatially arranged in a 2D planar array. The detecting antibody against a target compound may be the same as the capturing antibody against the same target compound.

In Step 305, aimed to be completed within one minute, upon the removal of the magnetic field, Reagent F may be removed along with large and small superparamagnetic beads that are not attached to the solid surface and an image of the solid surface may be captured with a camera of a smartphone and then processed by a smartphone app to produce result.

Reagent D may be a buffered solution containing large superparamagnetic beads covered with one or more capturing antibodies and small superparamagnetic beads covered with one or more target compounds. Each large superparamagnetic bead may be attached with one capturing antibody that is specific to a target compound listed in Tables 1 and 2 in the 2021 ANSI/ASB Standard for the Analytical Scope and Sensitivity of Forensic Toxicological Testing of Blood in Medicolegal Death Investigations. Each small superparamagnetic bead may be attached with a target compound listed in Tables 1 and 2 in the 2021 ANSI/ASB Standard for the Analytical Scope and Sensitivity of Forensic Toxicological Testing of Blood in Medicolegal Death Investigations. The operation described in Step 301 may occur outside the test chamber of a test cartridge.

In Step 302, after postmortem whole blood is mixed with Reagent E in a partially filled test chamber of a test cartridge (FIG. 2, left image), target compounds in the whole blood may compete against small superparamagnetic bead-bound target compounds that are captured by corresponding capturing antibodies on large superparamagnetic beads. The test chamber may be only partially filled so that Reagent E won't reach the inner top flat surface of the test chamber. Movement of Magnet X may facilitate target compound competition by creating superparamagnetic bead movement within Reagent E while the test cartridge remains motionless.

Once Step 302 is completed, Magnet X immobilizes superparamagnetic beads on a surface of the test chamber while the liquid may be completely drained from the chamber into the waste container. Subsequently in Step 303, Reagent B, which is a wash solution to remove unbound molecules from the test chamber and from superparamagnetic beads may be injected into the chamber to wash over the partially filled test chamber and superparamagnetic beads. The test chamber may be again only partially filled so that Reagent B won't reach the inner top flat surface of the test chamber. The resuspension of superparamagnetic beads in Reagent B may be facilitated by Magnet X. The superparamagnetic beads may be washed by Reagent B more than once before they may be resuspended in Reagent F.

Reagent F may be a buffered solution to facilitate antibody-target compound binding. In Step 304, the test chamber may be fully filled with Reagent F (FIG. 2, right image). While both large and small superparamagnetic beads may be suspended in Reagent F, only small superparamagnetic beads that are detached from large superparamagnetic beads may reach the inner top flat surface of the test chamber because a mesh installed horizontally in the middle of the test chamber may prevent large superparamagnetic beads from reaching the inner top flat surface of the test chamber. Magnet Y, which is on top of the test cartridge but positioned at a distance to the test chamber, may move across the top surface to draw small superparamagnetic beads to the inner top flat surface of the test chamber. Lateral movement of Magnet Y may facilitate the local enrichment of small superparamagnetic beads near detecting antibodies immobilized on designated locations in a spatial 2D array on the inner flat surface of the test chamber. Target compounds that are attached to superparamagnetic beads may bind to corresponding detecting antibodies attached to the top inner flat surface of the test chamber. As a result, corresponding superparamagnetic beads may be attached at the locations for corresponding detecting antibodies.

In Step 305, Magnet Y may be moved back to its original position and Reagent F may be withdrawn completely from the test chamber along with unbound superparamagnetic beads. The number of small superparamagnetic beads retained at designated locations of the solid surface may correlate in a qualitative, semi-quantitative, or quantitative manner with the level of corresponding target compound in postmortem whole blood. The presence of bound beads darkens the corresponding positions in the 2D array and the resulting 2D pattern of light/dark mosaic may be subsequently captured by a camera of a smartphone and analyzed by an image processing algorithm implemented in a smartphone app. A test cartridge may have multiple test chambers so that multiple samples can be tested concurrently, or a single sample can be tested sequentially with different sets of antibodies to minimize cross-reactivity and potential conflict of binding conditions among antibodies.

The reagents used in the competitive workflow may be the same or similar to those used in the non-competitive workflow. For example, Reagent E may be similar or the same as Reagent A. Reagent F may be similar or the same as Reagent C. Reagent A may be similar or the same as Reagent C. In the competitive workflow, an antibody against a target compound may serve two purposes in two different steps. In Step 301, it serves as the capturing antibody on the surface of large superparamagnetic beads. In Step 304, it serves as the detecting antibody on a solid surface. While the competitive workflow may address the difficulty to developed matched antibody pair against small molecules, the use of a single antibody instead of a pair of antibodies to detect a target compound may result in weaker specificity in comparison with the non-competitive workflow, in which a pair of antibodies may be used to detect a target compound.

In Step 304 of the competitive workflow, only small superparamagnetic beads released from the surface of large superparamagnetic beads may be allowed to be in close contact with solid surface-bound detecting antibodies to minimize false positive signal resulted from the binding of small superparamagnetic beads who are still attached to large superparamagnetic beads to solid surface-bound detecting antibodies. A mesh inside the test chamber with hole size that is smaller than the diameter of large superparamagnetic beads but larger than small superparamagnetic beads may achieve the necessary separation by blocking large superparamagnetic beads while allowing small superparamagnetic beads to pass through, guided by a magnet on top of the solid surface spotted with detecting antibodies.

A magnetic field is applied to a solution to accelerate the formation of a complex of target compound and corresponding detecting antibody. The magnetic field may be of any type and may be created through electromagnetic mechanism, using a magnet, such as a neodymium magnet, or other conventional techniques known in the art. The magnetic field may be applied to the solid surface, either in a sweeping mechanism across the surface horizontally or vertically, or in a uniform fashion over the entire surface. Preferably, the magnetic field may be applied horizontally. The application of a magnetic field may cause the probe to move towards and/or move cross over the solid surface to encounter the biomarker immobilized to the solid surface. The magnetic field may be applied for a period of time sufficient for the target compound and corresponding detecting antibody to form a complex, for example, no more than about 5 minutes, 1 minute, or 30, 10, 5, 1, 0.5 or 0.1 seconds, preferably no more than about 1 second.

A test cartridge may have one or more test chambers. A test cartridge with a single test chamber either partially or fully filled with reagents is shown in FIG. 2. The test cartridge may have a round bottom to avoid tight corner that traps superparamagnetic beads and to accommodate the circular movement of magnet X. It may have a flat lid on top to accommodate the lateral movement of magnet Y and the optical image capture of signal. The inner surface of the test cartridge lid may be spotted with a 2D planar array of detecting antibodies against compounds to be screened.

The non-competitive workflow may require a pair of antibodies for each target compound or target compound class to be detected. Each antibody in a pair may recognize non-overlapping epitopes of the corresponding antigen in order to sandwich the antigen. Aptamers may be screened using the SELEX process as replacements of one or both antibodies in a matched antibody pair for a target compound.

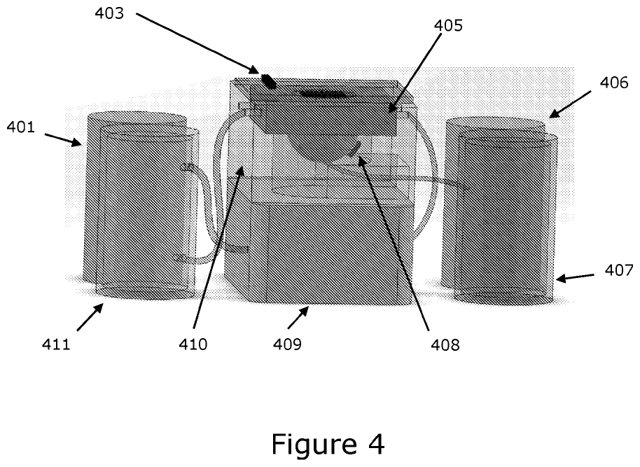

In another embodiment, detection of target compounds in a sample may be carried out in a lightweight portable tabletop device. The device may comprise the following components, some of which are shown in FIG. 4: A slot (410) to accept a test cartridge (405), three reagent containers (411, 401, and 406) for Reagents with tubing connected to the test cartridge through the slot, a waste container (407) with a tubing connected to the test cartridge through the slot, two mechanical arms driven by motors to move Magnet X (408) and Magnet Y (403) that are attached to the mechanical arms, a syringe pump (409) with four channels, three for the injection of three reagents and one for the removal of liquid from the test chamber, a smartphone slot to accept a smartphone, a smartphone, a custom-made PCB with onboard LED, a switch to start the screening process, and an enclosure.

The device may comprise four major subsystems in the device: (1) the microfluidic subsystem handles the sequential injection of three reagents into the test chamber and the removal of liquid from the test chamber. Controlled by the electronic subsystem, the syringe pump injects Reagents from the corresponding reagent containers into the test chamber at the corresponding steps. The syringe pump is also responsible to inject air into the test chamber to completely remove liquid from the test chamber and into the waste container. The size of tubing and the relative position of the test cartridge in regard to the waste container ensures that liquid won't be emptied from the test chamber into the waste container without the action of the syringe pump; (2) the mechanical subsystem comprises a servo or DC motor with a custom cam, controlled by the electronic subsystem, drives the syringe pump. It further comprises two additional motors that drive two mechanical arms with attached magnets X and Y so that movements of both magnets are coordinated with the movement of liquid; (3) the electronic subsystem comprises a custom designed PCB loaded with custom firmware that controls the entire operation triggered by a switch. It coordinates the movement of magnets and the movement of liquid. It further comprises an on-board LED provides necessary lighting for the optical detection of signal; (4) the optical detection subsystem comprises a smartphone slot and a smartphone with a custom-made app that takes a picture of a flat surface of the test cartridge and processes the image into semi-quantitative test results. It further comprises a macro lens that is embedded in the smartphone slot to optimize feature size in captured image.

The signal of the complexes of target compound, bound probes, and magnetic label may be then detected using various means. Many signal detection technologies routinely used in biomedical research and development, such as fluorescent labeling, colorimetric labeling, luminescent labeling, and magnetic labeling, may be implemented in signal detection. Surface plasmon resonance and bio-layer interferometry may also be considered as alternatives. Preferably, the complex is detected visually. A signal detection module may be used to detect colorimetric labeling. The signal detection module may use a camera such as the one on a cell phone to capture the image of the solid surface, process the image using an imaging processing software, and translate the signal into data in digital format. The data may then be sent to a data processing unit, which may be either as a part of a device or at a remote location through wired or wireless connection. Result may then be sent back to the data processing unit, which may in turn display such information to the user.

In some embodiments, a magnetic signal detection module may comprise an array of giant magnetoresister (GMR) sensors that matches the array of detecting antibodies on the inner flat surface of a test chamber (FIG. 2). The presence or absence or the quantity of a complex of target compound, probe, and magnetic label may induce different GMR effect, which is captured by an integrated circuit board, compared with reference sensors, and interpreted by software.

The fast target compound detection method of the present invention may be used for various purposes. For example, it may be used for postmortem toxicology screening, routine employment-related drug screening, blood stream infection detection, waste water monitoring. It may also be used in a diagnostic test to detect the presence of small molecule contaminants, pathogens, biological weapons, tumor antigens, or biomarkers. It may improve the performance of ELISA assay.

For each detection method of the present invention, a kit is provided for carry out the method. The kit may comprise the probe having a magnetic label and the solution.

For each detection method of the present invention, a device is provided for carry out the method. The device may comprise an electronic subsystem, a mechanical subsystem, a microfluidic subsystem, and a detection subsystem.

The term “about” as used herein, when referring to a measurable value such as an amount, a percentage, and the like, is meant to encompass variations of ±20%, ±10%, more preferably ±5%, even more preferably ±1%, and still more preferably ±0.1% from the specified value, as such variations are appropriate.

Example 1. Establish the sensitivity and specificity of the fast, semi-quantitative, array-based multiplex immunoassay for postmortem toxicology screening using the non-competitive workflow

First, Dynabeads MyOne Streptavidin C1 beads (Thermo Fisher Scientific, NJ) that have been incubated with 0.1 ug of Biotin-RM 428, an anti-25OH Vitamin D3 antibody (RevMAb Biosciences, CA), washed, and resuspended in 100 ul antibody binding buffer are split into two sets of tubes (Set A and Set B), each with half of the beads. Subsequently, human whole blood spiked with various final concentrations (0 ng/ml, 5 ng/ml, 10 ng/ml, 20 ng/ml, 50 ng/ml, 100 ng/ml, 200 ng/ml) of 25-OH Vitamin D3 is added to both set of the tubes. In addition, Fentanyl and Risperidone are added to tubes in Set B to reach final concentration of 10 ng/ml or 200 ng/ml, respectively so that the presence of Fentanyl and Risperidone on the sensitivity of 25-OH Vitamin D3 can be evaluated. After incubation and wash, beads in each tube in Set A and Set B are loaded into individual incubation chambers and drawn to the surface of glass slides spotted with detection antibody RMH04, another anti-25OH Vitamin D3 antibody (RevMAb Biosciences, CA). The number of magnetic beads attached at each spot will be quantified. Similar experiments to evaluate the effect of the other two target compounds on Fentanyl or Risperidone will be carried out. The concentration series of 0 ng/ml, 0.5 ng/ml, 1 ng/ml, 2 ng/ml, 5 ng/ml, 10 ng/ml, 20 ng/ml will be used for Fentanyl, while the concentration series of 0 ng/ml, 50 ng/ml, 100 ng/ml, 200 ng/ml, 500 ng/ml, 1000 ng/ml, 2000 ng/ml will be used for Risperidone.

Second, Dynabeads MyOne Streptavidin C1 beads functionalized with capturing antibody against either 25-OH Vitamin D3, Fentanyl, or Risperidone will be mixed together in human whole blood with 25-OH Vitamin D3, Fentanyl, or Risperidone at various concentrations described in the previous experiment. The bead mixture will be applied to the surface of a glass slide on which detection antibody against 25-OH Vitamin D3, Fentanyl, or Risperidone is spotted at specified locations of a 2D array at various concentrations so that sensitivities to detect all three target compounds in the same multiplex assay can be established.

Third, other compounds that are similar to 25-OH Vitamin D3, Fentanyl, or Risperidone will be evaluated individually or in combination in the non-competitive workflow to establish (1) the specificity of the non-competitive workflow for Fentanyl; (2) the specificity of the non-competitive workflow for Risperidone; and (3) the specificity of the multiplex non-competitive workflow for all three target compounds. The other compounds to be tested include acetylefentanyl, norfentanyl, risperidone, oxycodone, Vitamin D3, Vitamin D2, and 25-OH Vitamin D2. Additional compounds may be tested.

Example 2. Establish the sensitivity and specificity of the fast, semi-quantitative, array-based multiplex immunoassay for postmortem toxicology screening using the competitive workflow

A glass slide is cleaned before MH004 (Medix Biochemica Group, MO), a monoclonal antibody against Fentanyl, is diluted to 0 ug/ml, 1 ug/ml, 2 ug/ml, 10 ug/ml, and 20 ug/ml in the coating buffer then spotted on the glass slide in three duplicated rows to form a 2D array. The glass slide is then covered with an incubation chamber (Grace Biolabs) and incubated overnight at 4° C. Subsequently, the glass slide is washed with the wash buffer then with the antibody binding buffer according to protocol.

20 ul of 10 mg/ml Streptavidin-coated EPRUI-MagSA-10 beads (EPRUI Biotech, China) are washed in 30 ul biotin binding buffer then incubated with 0.1 ug of biotinylated MH 004 for 15 minutes in the biotin binding buffer. The beads are washed in the biotin binding buffer then in the antibody binding buffer. 20 ul of 10 mg/ml Dynabeads MyOne Carboxylic beads (Thermo Fisher Scientific, NJ) are incubated with 100 ng/ml Fentanyl-BSA conjugate and carbodiimide in buffer provided by the manufacturer according to the one-step protocol, allowing Dynabeads MyOne Carboxylic beads to be coated with Fentanyl-BSA conjugate. Subsequently Dynabeads MyOne Carboxylic beads are washed in the antibody binding buffer.

EPRUI beads and Dynabeads MyOne Carboxylic bead are then combined in solution to allow smaller Dynabeads MyOne Carboxylic beads (1 um in diameter) bind to the surface of larger EPRUI beads (10 um in diameter) through Fentanyl-MH004 binding. Beads are examined under a microscope to evaluate the completeness of coverage by Dynabeads MyOne Carboxylic beads on the surface of EPRUI beads. Binding condition will be optimized. A mesh is used to isolate EPRUI beads from unbound Dynabeads MyOne Carboxylic beads. The isolated EPRUI beads, some or all carrying one or more Dynabeads MyOne Carboxylic beads, are resuspended in 100 ul of the antibody binding buffer and Fentanyl is added to the solution to achieve final concentration of 0 ng/ml, 0.5 ng/ml, 1 ng/ml, 2 ng/ml, 5 ng/ml, 10 ng/ml, or 20 ng/ml. After incubating for 20 minutes, during which some Dynabeads MyOne Carboxylic beads are released from the surface of EPRUI beads due to competition from Fentanyl in solution, the beads are washed and resuspended in the antibody binding buffer. Afterwards EPRUI beads are separated from released Dynabeads MyOne Carboxylic beads using a mesh.

Dynabeads MyOne Carboxylic beads that are released and separated from EPRUI beads are loaded into the incubation chamber on the glass slide. Binding of Dynabeads MyOne Carboxylic beads to the surface of glass slide is facilitated with a magnet that moves across the solid surface of the glass slide on the opposite side. At selected time interval such as zero minute, one minute, 2 minutes, 5 minutes, 10, minutes, 30 minutes, and 60 minutes, the solid surface will be examined with naked eyes and under a microscope to evaluate the amount of Dynabeads MyOne Carboxylic beads that are bound to the solid surface at various locations where MH004 is spotted. Images will be taken with a camera under the microscope at specified time intervals and be processed to estimate the number of beads that are bound to solid surface at specified time intervals in order to create a sensitivity curve for Fentanyl in buffer solution under the competitive workflow. Images will also be taken with a camera behind a macro lens.

Once the competitive workflow is worked out with a pure solution containing Fentanyl, the solution will be replaced with human whole blood spiked with various final concentrations (0 ng/ml, 0.5 ng/ml, 1 ng/ml, 2 ng/ml, 5 ng/ml, 10 ng/ml, or 20 ng/ml) of Fentanyl to establish a sensitivity curve for Fentanyl in whole blood with the competitive workflow.

Similar experiments to obtain sensitivity curves for Risperidone, Oxycodone, and Amphetamine will be conducted. Subsequently, spiked whole blood with all four target compounds and with four corresponding EPRUI beads carrying Dynabeads MyOne Carboxylic beads are tested in a multiplex competitive workflow to establish the final sensitivity curves for those four target compounds.

To establish specificity, known cross-reactive compounds will be evaluated individually or in combination in the competitive workflow to establish (1) the specificity of the competitive workflow for individual target compound: Fentanyl, Risperidone, Oxycodone, and Amphetamine; and (2) the specificity of the multiplex competitive workflow for all four target compounds. The compounds to be tested include acetylefentanyl, norfentanyl, oxymorphone, noroxymorphone, noroxycodone, codeine, hydromorphone, risperidone, oxycodone, I-Methamphetamine, MDA, MDEA. Additional compounds may be tested.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed is:

1. A method for detecting one or more target compounds in a sample, the method comprising:
mixing a sample with a first solution that contains magnetic labels covered with one or more capturing probes, the one or more capturing probes being specific to one or more target compounds to be detected in the sample, the one or more capturing probes being immobilized exclusively on individual magnetic labels of the magnetic labels;
forming, in the first solution, complexes of the one or more target compounds and the one or more corresponding capturing probes;
washing the magnetic labels in a second solution;
mixing the magnetic labels in a third solution;
applying a magnetic field to bring the magnetic labels in proximity with one or more detecting probes, the one or more detecting probes being specific to one or more target compounds to be detected in the sample, to form one or more super-complexes of detecting probe and corresponding existing complexes of capturing probe and target compound;
removing the magnetic field;
isolating the super-complexes; and
detecting the presence of the super-complexes.

2. The method of claim 1, further comprising:
providing a surface with the one or more detecting probes attached thereto.

3. The method of claim 2, wherein the one or more detecting probes are arranged in a planar array.

4. The method of claim 1, wherein the one or more detecting probes include antibodies.

5. The method of claim 1, wherein the one or more capturing probes include aptamers.

6. The method of claim 1, wherein the detecting comprises capturing an image with an image capture device.

7. The method of claim 1, wherein one or more target compounds are under the drug classes of barbiturates, benzodiazepines, cannabinoids, dissociatives, cocaine, opioids, and sympathomimetics amines.

8. The method of claim 1, wherein the sample includes whole blood.

9. The method of claim 1, wherein detecting includes at least one of detecting at least one of fluorescence, luminescence, giant magnetoresistance (GMR), and color.

10. The method of claim 1, wherein the one or more complexes include at least one of the binding combinations biotin-streptavidin, antigen-antibody, and nucleotide double helix.

11. A method for detecting one or more target compounds in a sample, the method comprising:
mixing in a first solution large magnetic labels covered with one or more capturing probes, the one or more capturing probes being specific to one or more target compounds to be detected in the sample, the one or more capturing probes being immobilized exclusively on individual large magnetic labels of the large magnetic labels, with small magnetic labels covered with one or more target compounds to be detected in the sample, the one or more target compounds being immobilized exclusively on individual small magnetic labels of the small magnetic labels;
forming, in the first solution, large magnetic labels whose surface is covered with small magnetic labels;
mixing a sample in a second solution containing large magnetic labels covered with small magnetic labels, wherein target compounds in the sample compete with target compounds on the small magnetic labels to bind to capturing probes on the large magnetic labels to release into solution the corresponding small magnetic labels from the large magnetic labels;
washing the large and small magnetic labels in a third solution;
mixing the large and small magnetic labels in a fourth solution;
filtering the large magnetic labels from the released small magnetic labels;
applying a magnetic field to bring the small magnetic labels in proximity with one or more detecting probes to form one or more complexes of detecting probe and corresponding target compound;
removing the magnetic field;
isolating the super-complexes; and
detecting the presence of the super-complexes.

12. The method of claim 11, further comprising:
providing a surface with the one or more detecting probes attached thereto.

13. The method of claim 12, wherein the one or more detecting probes are arranged in a planar array.

14. The method of claim 11, wherein the one or more detecting probes include antibodies.

15. The method of claim 11, wherein the one or more capturing probes include aptamers.

16. The method of claim 11, wherein the detecting comprises capturing an image with an image capture device.

17. The method of claim 11, wherein one or more target compounds are under the drug classes of barbiturates, benzodiazepines, cannabinoids, dissociatives, cocaine, opioids, and sympathomimetics amines.

18. The method of claim 11, wherein detecting includes at least one of detecting at least one of fluorescence, chemiluminescence, giant magnetoresistance (GMR), and color.

19. The method of claim 11, wherein the one or more complexes include at least one of the binding combinations biotin-streptavidin, antigen-antibody, and nucleotide double helix.

20. A system for detecting one or more target compounds in a sample, the system comprising:
a microfluidic subsystem configured to mix a sample with a first solution that contains magnetic labels covered with one or more capturing probes, the one or more capturing probes being specific to one or more target compounds to be detected in the sample, the one or more capturing probes being immobilized exclusively on individual magnetic labels of the magnetic labels, form, in the first solution, complexes of the one or more target compounds and the one or more corresponding capturing probes, wash the magnetic labels in a second solution, and mix the magnetic labels in a third solution;
a magnetic field generator configured to bring the magnetic labels in proximity with one or more detecting probes, the one or more detecting probes being specific to one or more target compounds to be detected in the sample, to form one or more super-complexes of detecting probe and corresponding existing complexes of capturing probe and target compound;
the microfluidic subsystem configured to isolate the super-complexes; and
a detection subsystem configured to determine the presence and quantity of the super-complexes.
